(19) United States
(12) Patent Application Publication
(10) Pub. No.: US 2004/0152921 A1
(43) Pub. Date: Aug. 5, 2004

(54) Conversion of carbon disulfide and hydrogen to methyl mercaptans

(75) Inventors: Guido Mul, Nootdorp (NL); Albert S. Hirschorn, Menlo Park, CA (US); Robert B. Wilson JR., Palo Alto, CA (US)

Correspondence Address: BANNER & WITCOFF, 1001 G STREET N W, SUITE 1100, WASHINGTON, DC, 20001, US

(73) Assignee: Georgia-Pacific Resins, Inc., Atlanta, GA (US)

(21) Appl. No.: 10/701,171

(22) Filed: Nov. 5, 2003

Related U.S. Application Data
(60) Provisional application No. 60/424,011, filed on Nov. 6, 2002.

(51) Int. Cl. C07C 319/02

(52) U.S. Cl. 568/69

(57) ABSTRACT

Carbon disulfide and hydrogen are reacted in the presence of a catalyst to form methyl mercaptans. The catalyst may be V₂O₅, Re₂O₇, or MnO supported on a substrate of CeO₂, ZrO₂, TiO₂, Nb₂O₅, Al₂O₃, SiO₂, Ta₂O₅, SnO₂, or mixtures thereof High conversions of carbon disulfide and high selectivities to methanethiol are obtained by the present invention.

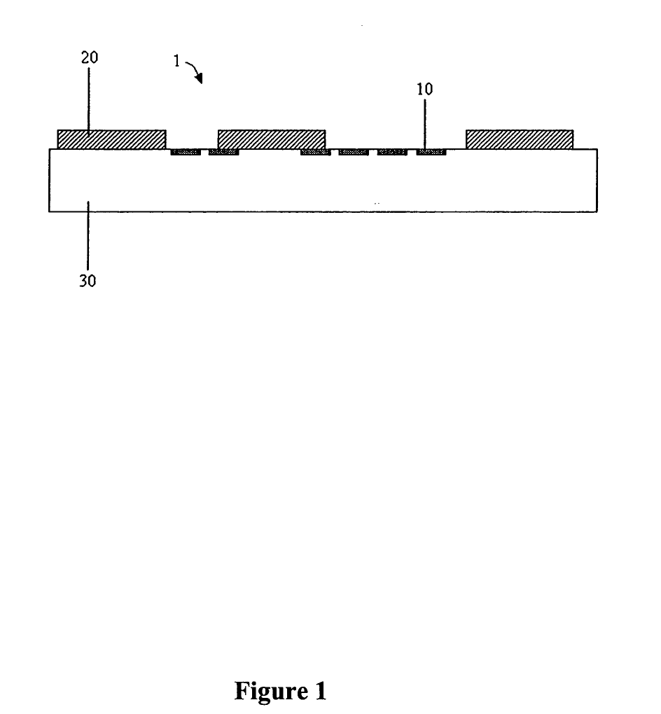

### FIELD OF THE INVENTION

This invention relates to the treatment of sulfur-containing process streams, and more particularly to the catalytic conversion of carbon disulfide and hydrogen to methyl mercaptans.

### BACKGROUND OF THE INVENTION

Carbonyl sulfide (COS), hydrogen sulfide (H₂S), methane (CH₄), and carbon disulfide (CS₂) are typical by-products produced from processing streams containing sulfur compounds, such as encountered in the pulp mill, petroleum, natural gas, and steel industries. Carbonyl sulfide and carbon disulfide can be readily hydrolyzed by steam to form CO₂ and H₂S, and H₂S may be converted to elemental sulfur by the Claus Process:

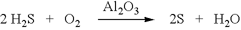

It also is often desirable to convert sulfur-containing compounds into more valuable chemical intermediates such as methyl mercaptans, e.g., methanethiol (CH₃SH), dimethyl sulfide (CH₃SCH₃) and dimethyl disulfide (CH₃SSCH₃). Several methods have been proposed for the preparation of mercaptans. For example, van Venrooy, U.S. Pat. No. 3,488,739, describes the preparation of methanethiol and dimethyl sulfide by passing carbon disulfide and excess hydrogen over a supported sulfur tolerant hydrogenation catalyst. The catalyst may be a sulfide of Group VI and VIII metals, such as cobalt, nickel, molybdenum, tungsten, chromium, platinum, or combinations thereof. Catalytic supports include activated carbon, alumina, zirconia, thoria, pumice, silica and silica-aluminum compositions.

Kubicek, U.S. Pat. No. 3,880,933, discloses a process for the conversion of carbon disulfide to methanethiol by hydrogenation in the presence of a sulfur tolerant hydrogenation catalyst and hydrogen sulfide. As in van Venrooy, the catalysts are sulfides of Group VI and VIII metals, such as cobalt, nickel or molybdenum and combinations thereof. Catalytic supports include activated carbon, alumina, zirconia, thoria, pumice, silica and silica-aluminum compositions.

Chang, U.S. Pat. No. 4,543,434, and Audeh, U.S. Pat. No. 4,882,938, describe methods for converting methane to higher molecular weight hydrocarbons via sulfur-containing intermediates. Chang discloses the following reaction steps:

CH₄+4 S→CS₂+2 H₂S 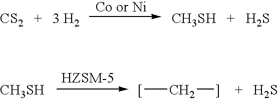
4 H₂S→H₂+4 S

where [—CH₂—] represents one or more saturated hydrocarbons having at least two carbon atoms. Audeh discloses that methanethiol may be generated by a reaction between methane, carbon disulfide, and hydrogen having the following stoichiometry:

CH₄+CS₂+2H₂→2CH₃SH

Audeh theorizes that the reaction could take place by the following two-step mechanism:

CS₂+2 H₂→CH₃SH+S

S+CH₄→CH₃SH

Audeh teaches that the reaction of methane with carbon disulfide and hydrogen may take place in the presence or absence of hydrogen sulfide. The hydrogen sulfide, when present, may react with the methane and/or carbon disulfide under certain (undisclosed) conditions. Other organosulfur compounds may be formed, such as dimethyl sulfide, from the reaction of methane, carbon disulfide, and hydrogen. Audeh teaches that the reaction between methane, hydrogen, and carbon disulfide may occur at a temperature from about 25° C. to about 500° C. and a pressure of from about 1 atmosphere to about 200 atmospheres, but does not teach the use of catalysts for the reaction. According to Audeh, the organosulfur compounds may then be contacted with a zeolite catalyst to produce higher molecular weight hydrocarbons and hydrogen sulfide.

### SUMMARY OF THE INVENTION

It is an object of the present invention to develop an alternative cost-effective process for converting carbon disulfide and hydrogen to methyl mercaptans.

According to the present invention, a process for producing methyl mercaptans comprises contacting a stream comprising carbon disulfide and hydrogen with a supported metal oxide catalyst under conditions sufficient to form methyl mercaptans. The catalyst is selected from the group consisting of V₂O₅, Re₂O₇, and MnO, and is supported on a substrate selected from the group consisting of CeO₂, ZrO₂, TiO₂, Nb₂O5, Al₂O₃, SiO₂, Ta₂O₅, SnO₂, and mixtures thereof.

High conversions of CS₂ and high selectivities to CH₃SH may be obtained by the reactions of the present invention.

### BRIEF DESCRIPTION OF THE DRAWING

The present invention will now be described in more detail with reference to preferred embodiments of the invention, given only by way of example, and illustrated in the accompanying drawing in which:

the FIGURE is a schematic illustration of a catalyst supported on a substrate according to a preferred embodiment of the invention.

### DETAILED DESCRIPTION OF THE INVENTION

The FIGURE schematically illustrates a supported metal oxide catalyst 1 in accordance with the present invention. The metal oxide of the supported metal oxide catalyst is accommodated on the support primarily as a two-dimensional metal oxide overlayer, with the oxide having a non-crystalline form. The catalyst 1 may comprise a vanadium sulfide phase 20 on a titania support 30. As the reaction proceeds (i.e., from left to right in the FIGURE), portions 10 of the titania surface may also become sulfided.

The general reaction of the invention is expressed by the equation:

CS₂+3 H₂⇄CH₃SH+H₂S

Pressure drives the reaction to the right, since four moles of reactants form two moles of product. While not wanting to be bound by theory, it is believed that the following reaction mechanism occurs:

CS₂ (g)+½ H₂ (g)⇄HCS₂ (ads)

HCS₂ (ads)+2 H₂ (g)⇄CH₃S (ads)+H₂S (g)

CH₃S (ads)+½H₂ (g)⇄CH₃SH (g)

wherein (ads)=surface intermediate on catalyst, and (g)=gas phase molecule.

As used herein, the term “selectivity,” for example the selectivity of methanethiol, is determined by dividing the number of moles of methanethiol formed by the number of moles of carbon disulfide consumed from the reactant gas feed stream times 100. Accordingly, selectivity is a percentage value. Selectivity indicates the percentage of methanethiol formed as compared to the percentage of non-methanethiol carbon products of the reaction such as CH₄, C₂H₆, etc. Unless otherwise indicated, the selectivity of methanethiol, as used herein, is based on the number of moles of methanethiol formed plus 2 times the number of moles of dimethylsulfide (DMS) formed, on the assumption that DMS is formed by the reaction of 2 molecules of methanethiol.

As used herein, the term “conversion,” for example the conversion of carbon disulfide, is determined by dividing the difference between the number of moles of carbon disulfide fed to the reactor in the reactant gas feed stream minus the number of moles of carbon disulfide exiting the reactor by the total number of moles of carbon disulfide fed times 100. Accordingly, conversion is also a percentage value. Conversion indicates the percentage of the moles of carbon disulfide that were converted to methanethiol or any other reaction products.

Thus, if 2 moles of carbon disulfide are fed into the reactor (e.g., in a reactant gas feed stream) yielding 1 mole of methanethiol and 1 mole of carbon disulfide, then selectivity would equal 100% while conversion would equal 50%. Likewise, if 3 moles of carbon disulfide are fed into the reactor (e.g., in a reactant gas feed stream) yielding 2 moles of methanethiol and 1 mole of carbon disulfide, then selectivity would equal 100% while conversion would equal 66 and ⅔%.

The efficiency of the desired reactions is enhanced by the use of reducible metal oxide catalysts which easily become sulfided under the reaction conditions set forth herein. The catalyst may be V₂O₅, Re₂O₇, or MnO supported on a substrate of CeO₂, ZrO₂, TiO₂, Nb₂O₅, Al₂O₃, SiO₂, Ta₂O₅, SnO₂, or mixtures thereof. The most preferred catalyst is vanadia (V₂O₅) supported on titania (TiO₂). These supported metal catalysts are commercially available and/or readily prepared by those skilled in the art. See, e.g., WO 98/17618, incorporated by reference herein. Further details on the preparation and structure of such supported metal oxide catalysts useful in the practice of the present invention can be found in Jehng et al., Applied Catalysis A, 83, (1992) 179-200; Kim and Wachs, Journal of Catalysis, 142, 166-171; Jehng and Wachs, Catalysis Today, 16, (1993) 417-426; Kim and Wachs, Journal of Catalysis, 141, (1993) 419-429; Deo et al., Applied Catalysis A, 91, (1992) 27-42; Deo and Wachs, Journal of Catalysis, 146, (1994) 323-334; Deo and Wachs, Journal of Catalysis, 146, (1994) 335-345; Jehng et al., J. Chem. Soc. Faraday Trans., 91(5), (1995) 953-961; Kim et al., Journal of Catalysis, 146, (1994) 268-277; Banares et al., Journal of Catalysis, 150, (1994) 407-420 and Jehng and Wachs, Catalyst Letters, 13, (1992) 9-20, the disclosure of which are incorporated herein by reference.

The term “catalyst loading” used herein refers to weight of the active component of the catalyst (e.g., the weight of vanadia) divided by the total weight of the catalyst (e.g., the weight of vanadia plus the weight of titania support). Catalyst loading is thus a percentage value. Generally, the metal oxide loading on the metal oxide support or substrate broadly ranges between about 0.5 and about 35 wt %, with 1 to 25 wt % being more typical, and 1 to 10 wt % being used most often.

Hydrogen may be reacted with carbon disulfide over a catalyst to convert the reactants to methyl mercaptans. By-products may include H₂S, CH₄, and C₂H₆. Suitable exemplary reaction conditions include a space velocity of from about 0.01 to 1.5 g_{Cat} g_{CO}⁻¹ h⁻¹, more typically from about 0.05 to 1 g_{Cat} g_{CO}⁻¹ h⁻¹, and even more typically about 0.1 g_{Cat} g_{CO}⁻¹ h⁻¹; a catalyst loading of from about 2 to 6%, more typically from about 3 to 5%, and even more typically about 4%; a ratio of moles of H₂ to moles of CS₂ of from about 1:2 to 10:1, more typically from about 2:1 to 5:1, and even more typically about 3:1; a reaction pressure of from about 0.1 to 600 psig, more typically from about 50 to 300 psig, and even more typically from about 100 to 250 psig; a reaction temperature of from about 250 to 600° C., more typically from about 300 to 500° C., and even more typically from about 325 to 450° C.

Preferably, hydrogen and carbon disulfide are fed in a reactant stream at or in moderate excess of the stoichiometric ratio, i.e., 3:1. At lower H₂:CS₂ ratios, lower conversions of CS₂ are obtained. At higher H₂:CS₂ ratios, e.g., greater than 10:1, significant hydrogenation of the mercaptans may occur, undesirably consuming a large amount of the expensive hydrogen reactant and yielding methane via the following reactions:

CH₃SH+H₂→CH₄+H₂S

CH₃SCH₃+H₂→2CH₄+H₂S

Reaction pressure generally is not critical, although higher pressures may be advantageous, e.g., by enabling lower reaction temperatures to be employed. Temperature generally is more critical. At atmospheric pressure, the reaction predominately occurs within the temperature range of 300° C. to 500° C., especially 325° C. to 450° C.

It will be apparent to those skilled in the art that various modifications and variations can be made in the compositions and methods of the present invention without departing from the spirit or scope of the invention. Thus, it is intended that the present invention cover the modifications and variations of this invention provided they come within the scope of the appended claims and their equivalents.

What is claimed is:

1. A process for producing methyl mercaptans, the process comprising contacting a stream comprising carbon disulfide and hydrogen with a catalyst under conditions sufficient to form methyl mercaptans, wherein said catalyst is selected from the group consisting of V₂O₅, Re₂O₇, and MnO, and wherein said catalyst is supported on a substrate selected from the group consisting of CeO₂, ZrO₂, TiO₂, Nb₂O₅, Al₂O₃, SiO₂, Ta₂O₅, SnO₂, and mixtures thereof.

2. The process of claim 1 wherein said conditions include a temperature of from about 250° C. to about 600° C.

3. The process of claim 2 wherein said temperature is from about 300° C. to about 500° C.

4. The process of claim 2 wherein said temperature is from about 325° C. to about 450° C.

5. The process of claim 1 wherein said stream comprises hydrogen and carbon disulfide at a molar ratio of from about 1:2 to about 10:1.

6. The process of claim 5 wherein said molar ratio is from about 2:1 to about 5:1.

7. The process of claim 1 wherein said catalyst comprises V₂O₅ supported on TiO₂.

8. The process of claim 1 wherein said catalyst comprises V₂O₅ supported on Al₂O₃.
